(19) United States
(12) Patent Application Publication
(10) Pub. No.: US 2012/0076770 A1
(43) Pub. Date: Mar. 29, 2012

(54) Modulation of autophagy and and serotonin for treatment of multiple myeloma related diseases

(76) Inventors: Virginia Espina, Rockville, MD (US); Lance Liotta, Bethesda, MD (US); Antonella Chiechi, Matera (IT); Alessandra Romano, Catania (IT); Emanuel Petricoin, Gainesville, VA (US); Amy Van Meter, Bristow, VA (US)

(21) Appl. No.: 13/073,989

(22) Filed: Mar. 28, 2011

Related U.S. Application Data
(63) Continuation-in-part of application No. PCT/US2009/004608, filed on Dec. 8, 2009.
(60) Provisional application No. 61/318,074, filed on Mar. 26, 2010.

(51) Int. Cl. A61K 31/4706; A61K 31/436; A61K 31/404; A61K 31/506; A61K 31/675; A61K 39/395; A61P 19/08; A61P 35/00; A61K 31/663; A61K 31/353; A61K 31/517

(52) U.S. Cl. 424/130.1; 514/456; 514/291; 514/414; 514/252.19; 514/266.24; 514/313; 514/108; 514/94

(57) ABSTRACT

THE INVENTION RELATES TO compounds, proteins and methods of treatment therewith. Aspects of embodiments of the invention further relates to compounds and methods of treatment for bone, bone marrow, and bone tissue.

### CROSS-REFERENCE TO RELATED APPLICATIONS

This application claims the benefit of U.S. Provisional Application No. 61/318,074, filed 26 Mar. 2010, entitled “Modulation of Autophagy and Serotonin for Treatment of Multiple Myeloma Related Diseases,” and is a continuation-in-part (CIP) of PCT application PCT/US2009/004608, with an international filing date of 12 Aug. 2009, each of which is hereby incorporated by reference in its entirety.

### BRIEF DESCRIPTION OF THE SEVERAL VIEWS OF THE DRAWINGS

FIG. 1 is an artist's rendering of a physiological mechanism of Serotonin-based regulation of bone mass as per aspect of an embodiment of the present invention.

FIG. 2 depicts three graphs illustrating a relationship between Serotonin, the angiogenesis switch (HIF-1α), and bone lesions in patients treated with bone breakdown inhibitors (bisphosphonates) as per aspect of an embodiment of the present invention.

FIG. 3 reveals two graphs depicting the serum-Serotonin concentration and the platelet grains-Serotonin concentration for healthy, diseased, and disease-free bone tissue as per aspect of an embodiment of the present invention.

FIG. 4 is a multipanel figure depicting a correlation between Serotonin levels and RANK, DKK1, and cytokines that are known to regulate bone.

FIG. 5 contains two graphs depicting Serotonin's positive correlation with both IL-10 and TNF-α as per aspect of an embodiment of the present invention.

FIG. 6 is a graph comparing the Serotonin, RANK, lrp6, progest rec S190, beta-arrestin, and DEPTOR concentrations in diseased and healthy bone marrow aspirates as per aspect of an embodiment of the present invention.

### DETAILED DESCRIPTION OF EMBODIMENTS

Embodiments relate to methods of treating disease in subjects. Other embodiments relate to articles of manufacture useful in treating disease, methods of making therapeutic compositions, combinations of therapeutic compositions, methods of administering therapeutic compositions, and dosages of therapeutic compositions.

In an aspect of an embodiment, a process to treat myelodysplasia includes methods of treating monoclonal gammopathy of unknown significance (MGUS), processes to treat multiple myeloma, processes to treat bone dysplasia and processes to treat abnormalities in bone.

In one aspect of an embodiment, a process to treat monoclonal gammopathy of unknown significance (MGUS) may include administering to a patient a therapeutically effective amount of a 4-aminoquinoline compound and a tyrosine kinase inhibitor.

In one embodiment, a process to modulate bone remodeling in a subject may include modulating serotonin in a subject. In an additional embodiment, said process to modulate bone remodeling may include increasing or decreasing bone density.

In another embodiment, a process to modulate bone remodeling in a subject may include administering to a subject a serotonin modulator in an effective amount wherein serotonin levels in a subject are modulated. In a further teaching, a process to modulate bone remodeling produces plasma concentrations of platelet derived serotonin. In another aspect of an embodiment, a process to modulate bone remodeling in a subject may include administering to a subject a medicament in an effective amount wherein serotonin levels in a subject are modulated.

In still another embodiment a process for modulating bone dysplasia, treating monoclonal gammopathy of unknown significance (MGUS), or treating multiple myeloma may include administering a serotonin modulator to a subject in an effective amount. Said serotonin modulator may include a tyrosine kinase inhibitor, a selective serotonin reuptake inhibitor, a heterocyclic antidepressant, a monoamine oxidase inhibitor, an antidepressant, an anti-anxiolytic, an antiepileptic, or an antibody.

In an aspect of an embodiment, an article of manufacture may include at least one vessel containing purified chloroquine and purified HA14-1, instructions for the use of chloroquine and HA14-1 for the treatment monoclonal gammopathy of unknown significance (MGUS), multiple myeloma, bone dysplasia and/or abnormalities in bone, the treatment comprising (a) identifying a patient suspected of having said disease, and (b) administering an effective amount of chloroquine and HA14-1 to the patient.

In an aspect of an embodiment, an article of manufacture may include a label that indicates the contents of the package may be used to treat at least one of multiple monoclonal gammopathy of unknown significance (MGUS), multiple myeloma, bone dysplasia and/or abnormalities in bone, packaging material, and contained within the packing material purified chloroquine (or other 4-amino quinoline), and at least one of a purified tyrosine kinase inhibitor, a purified selective serotonin reuptake inhibitor (SSRI), a purified heterocyclic antidepressant, a purified monoamine oxidase inhibitor, a purified antidepressant, a purified anti-anxiety compound, a purified anti-epileptic, and a purified antibody.

In an aspect of an embodiment a method of treating bone disease may include administering a serotonin modulator to a subject. In an aspect of an embodiment, a method may include treating wherein said treating includes at least one of increasing bone density, decreasing bone density, maintaining bone density, or regulating elements associated with bone marrow.

An aspect of an embodiment a method of regulating may include altering at least one of preneoplastic differentiation of bone marrow cells, neoplastic angiogenesis of bone marrow cells, or bone marrow stem cell function.

In an aspect of an embodiment a method of regulating may include wherein serotonin modulator alter at least one of the ratio of serotonin in platelets to plasma, concentration of serotonin in the bone marrow, serotonin receptor activity of cells within the bone marrow, production of serotonin by cells associated with the bone, intracellular signaling pathways associated with serotonin.

In an aspect of an embodiment, a method of treating bone disease may include administering a serotonin modulator to a subject. In a further embodiment the method of treating bone disease may include wherein said treating comprises at least one of the following increasing bone density, decreasing bone density, maintaining bone density; and regulating elements associated with bone marrow.

A method of claim 2 where said regulating comprises altering at least one of the following: preneoplastic differentiation of bone marrow cells, neoplastic angiogenesis of bone marrow cells, or bone marrow stem cell function.

In an aspect of an embodiment, serotonin modulator may alter at least one of ratio of serotonin in platelets to plasma, concentration of serotonin in the bone marrow, serotonin receptor activity of cells within the bone marrow, production of serotonin by cells associated with the bone, intracellular signaling pathways associated with serotonin,

In an aspect of an embodiment a serotonin modulator may include a least one of a tyrosine kinase inhibitor, a selective serotonin reuptake inhibitor (SSRI), a heterocyclic antidepressant, a monoamine oxidase inhibitor, an antidepressant, an antianxiety compound, an antiepileptic compound, an antibody

In an aspect of an embodiment, a monoamine oxidase inhibitor may include a selective monoamine oxidase inhibitor, a monoamine oxidase A inhibitor, a monoamine oxidase B inhibitor or a nonselective monoamine oxidase inhibitor.

In an aspect of an embodiment, a method of treating bone disease may include administering at least one of an autophagy inhibitor, a non-chemotherapeutic agent, and angiogenesis inhibitor, a bone breakdown inhibitor, an osteoclast or osteoblast activity inhibitor, and an immune signal modulator.

An aspect of an embodiment, the method of treating bone disease may include reducing serotonin from platelet cells, gastrointestinal cells, neural cells, immune cells, bone marrow microenvironment cells or cancer cells.

In an aspect of an embodiment, a “bone breakdown inhibitor” is administered in an effective amount to modulate at least one of bone cell activity, stem cell activity, gastrointestinal cell activity, cancer cell activity, platelet cell activity, and neural cell activity.

In a further teaching, a method of treating bone disease includes bone diseases such as brittle bone disease, multiple myeloma, osteogenesis imperfecta, osteolytic bone disease, amyloidosis, monoclonal gammopathy, alterations in bone marrow hematopoetic precursor cells; and myelodysplasia.

In still another aspect, the method of treating bone disease encompasses treating subject at least one of a chordate, mammal, primate, and human. Another embodiment may include diagnosing and/or treating myeloma cells, including wherein said cells are inhibited, suppressed, or killed to a greater extent as compared to the non-myeloma cells.

Still another aspect of an embodiment includes a method of diagnosing a subject for a bone disease including at least the steps of assaying a biological sample of the subject, determining the amount of serotonin in said biological sample and determining a disease state based on said amount of serotonin.

Still another aspect of an embodiment includes diagnostic methods wherein said biological sample comprises at least one of the bone marrow aspirate, tissue, blood, serum, whole blood, cells; and blood.

Still another aspect of an embodiment includes diagnostic methods including at least the steps of determining the amount of serotonin in a known normal sample, determining the amount of serotonin in said biological sample, comparing said amount of serotonin in said known normal sample to the amount of serotonin in said biological sample. In another aspect of an embodiment, diagnostic methods include treating a subject based on serotonin level determination and or modulating serotonin in the subject based on said diagnosing.

In a still further teaching, diagnostic methods include assays evaluating post translational modification of signaling proteins, caspase cleavage, poly(ADP-ribose) polymerase (PARP) cleavage or dye exclusion/uptake.

According to another aspect of an embodiment, diagnostic methods include evaluating methods employing at least one of reverse phase microarray (RPMA), ELISA, flow cytometry, Immunohistochemistry, Immunoassay, high resolution mass spectroscopy, and suspension bead array.

In a further teaching, a method of treating monoclonal gammopathy of unknown significance (MGUS), premalignant bone marrow cells or multiple myeloma in a subject includes treating with an autophagy pathway inhibitor, and at least one of a tyrosine kinase inhibitor, a serotonin modulator, an antidepressant, an anti-anxiety compound, an antiepileptic, a monoamine oxidase inhibitor, an antibody, a non-chemotherapeutic agent and a bisphosphonate.

In a further teaching, an autophagy pathway inhibitor such as 4-amino quinoline may be used.

In a further aspect of an embodiment, treatment methods may retard the progression from a pre-disease state to multiple myeloma. In a further teaching, a modulator may include a lease one of a tyrosine kinase inhibitor; a selective serotonin reuptake inhibitor (SSRI); a heterocyclic antidepressant; a monoamine oxidase inhibitor; an antidepressant; an anti-anxiety compound; an anti-epileptic; and an antibody.

In a further embodiment, a monoamine oxidase inhibitor is a selective monoamine oxidase inhibitor, a monoamine oxidase A inhibitor, a monoamine oxidase B inhibitor or a nonselective monoamine oxidase inhibitor.

An additional aspect of an embodiment may include a method of treating bone disease including treating with at least one of an autophagy inhibitor; a non-chemotherapeutic agent; an angiogenesis inhibitor; a bone breakdown inhibitor; a osteoclast or osteoblast activity inhibitor; and an immune signal modulator.

In a further aspect of an embodiment, the method of treating bone disease may include reducing serotonin from at least one of platelet cells, gastrointestinal cells, neural cells, immune cells, bone marrow microenvironment cells or cancer cells.

In a further aspect of an embodiment, administering a bone breakdown inhibitor may include administering in an effective amount to modulate at least one of bone cell activity, stem cell activity, gastrointestinal cell activity, cancer cell activity, platelet cell activity, and neural cell activity. In a further aspect of an embodiment, a method of treating bone disease may include treating at least one of brittle bone disease; multiple myeloma; osteogenesis imperfecta (OI), osteolytic bone disease, amyloidosis; monoclonal gammopathy; alterations in bone marrow hematopoetic precursor cells; and myelodysplasia.

According to an aspect of an embodiment, the treatment methods may include treating at least one of a chordate, mammal, primate, and human.

According to embodiments, myeloma cells are inhibited, suppressed, or killed to a greater extent as compared to the non-myeloma cells.

According to embodiments, methods include selecting a subject in need of treatment.

According to embodiments, a method of diagnosing a subject for a bone disease includes assaying a biological sample of the subject; determining the amount of serotonin in said biological sample; and determining a disease state based on said amount of serotonin.

According to embodiments, a biological sample to be used in the method includes at least one of the following bone marrow aspirate; tissue; blood serum; whole blood; cells; and blood.

According to embodiments, diagnostic methods include determining the amount of serotonin in a known normal sample, determining the amount of serotonin in a biological sample, and comparing said amount of serotonin in said known normal sample to the amount of serotonin in said biological sample. According to embodiments, diagnostic methods further include treating a subject based on said determination.

According to embodiments, regulating bone remodeling in the subject includes modulating serotonin in the subject based on said diagnosing.

According to embodiments, the assaying step may include at least one of assays evaluating post translational modification of signaling proteins, caspase cleavage, poly(ADP-ribose) polymerase (PARP) cleavage or dye exclusion/uptake.

According to embodiments, diagnostic methods may include at least one or more of evaluating where the evaluating includes at least one of reverse phase microarray (RPMA), ELISA, flow cytometry, Immunohistochemistry, Immunoassay, high resolution mass spectroscopy, and suspension bead array.

According to embodiments, a method of treating monoclonal gammopathy of unknown significance (MGUS), premalignant bone marrow cells or multiple myeloma in a subject, includes administering an autophagy pathway inhibitor and at least one of a tyrosine kinase inhibitor, a serotonin modulator, an antidepressant, an anti-anxiety compound, an antiepileptic, a monoamine oxidase inhibitor, an antibody, a non-chemotherapeutic agent and a bisphosphonate.

In a further teaching, and autophagy pathway inhibitor is a 4-amino quinoline.

In a further teaching, a bisphosphonate may include at least one of alendronate, pamidronate or zoledronic acid.

In an aspect of an embodiment, the monoamine oxidase inhibitor may include a selective monoamine oxidase inhibitor, a monoamine oxidase A inhibitor, a monoamine oxidase B inhibitor or a nonselective monoamine oxidase inhibitor.

In an aspect of an embodiment, the method may include treating with an autophagy inhibitor, and angiogenesis inhibitor, a bone breakdown inhibitor, an osteoclast or osteoblast activity inhibitor, and in immune signal modulator.

In an aspect of an embodiment, a kit may include a vessel or vessels containing purified 4-amino quinoline (for example chloroquine) and at least one of at least one of a purified tyrosine kinase inhibitor, a purified selective serotonin reuptake inhibitor (SSRI), a purified heterocyclic antidepressant, a purified monoamine oxidase inhibitor, purified an antidepressant, a purified anti-anxiety compound, a purified anti-epileptic, and a purified antibody.

In a further teaching, a method of treating may include a route of administration wherein said route of the administration may include at least one of intramuscular, transdermally, transmucossally, rectally, orally, via nasal insufflation, intravenous administration, and via cerebrospinal fluid or lumbar injection.

According to an additional embodiment, the form of the medicament may include a lotion, patch, injectable, tablet, or nasal spray.

In an additional embodiment, chloroquine analogs include Chloroquine (CQ), 7-chloro-4-[[4-(diethylamino)-1-methylbutyl]amino]quinoline phosphate (1:2), Chloroquine Phosphate, USP, Qualiquin (Quinine), Plaquenil (hydroxychloroquine), Aralen, Aralen Phosphate, Lariam, or 4-aminoquinoline compounds.

According to embodiments, tyrosine kinase inhibitors include Azitinib, Bosutinib, Cediranib, Crizotinib, Damnacanthal, Dasatinib, Erlotnib, Gefitinib, Imatinib, Lapatinib, Lestaurtinib, Neratinib, Nilotinib, Regorafenib, Ruxolitinb, Semaxanib, Sunitinib, Toceranib, Tofacitinib, Vandetanib, or Vatalnib.

According to embodiments, serotonin modulators include selective serotonin reuptake inhibitors (SSRIs). SSRIs include Citalopram, Dapoxetine, Escitalopram, Fluoxetine, Fluoxetine, Paroxetine, or Sertraline.

Additional embodiments include tricyclic antidepressants (TCAs). TCAs include Amitriptyline, Butripyline, Clomipramine, Desipramine, Dawsey Letha, Doxepin, Nortriptyline, Protriptyline, or Trimipramine.

In another embodiment, monoamine oxidase inhibitors (MAOIs) include selective monoamine oxidase inhibitors, non-selective monoamine oxidase inhibitors, monoamine oxidase-A (MAO-A) inhibitors (Metralindole, Resveratrol, Berberine, Coptisine, Minaprine, Brofaromine, Toloxatone, Moclobemide, or Pirlindole), monoamine oxidase-B (MAO-B), inhibitors (Lazabemide, Pargyline, Rasagiline, or Selegiline), Hydrazines (Benmoxin, Hydralazine, Iproclozide, Iproniazid, Iprozid, Ipronid, Rivivol, Propilniazida, Isocarboxazid, Isoniazid, Mebanazine, Nialamide, Octamoxin, Phenelzine, Pheniprazine, Phenoxypropazine, Pivalylbenzhydrazine, Procarbazine, Natulan, or Indicarb, Safrazine), or Non-Hydrazines (Caroxazone, Echinopsidine, Furazolidone, Linezolid (Zyvox, Zyvoxam, Zyvoxid), or Tranylcypromine).

In another aspect, monoamine oxidase inhibitors include Valproic Acid, Diazepam, licorice, Siberian ginseng, Yerba Mate, or Yohimbe.

In another aspect, serotonin agonists include 5-HT_{1A} agonists (buspirone, gepirone, and tandospirone), 5-HT1B receptor agonists (sumatriptan, rizatriptan, and naratriptan), 5-HT_{1D} receptor agonists (sumatriptan, rizatriptan, and naratriptan), 5-HT_{1F} receptor agonist (Lasmiditan), 5-HT_{2A} receptor agonists (LSD, mescaline, psilocin, DMT, and 2C-B), 5-HT_{2c} receptor agonists (Lorcaserin), 5-HT₄ receptor agonist, and 5-HT₇ receptor agonists.

In another aspect, serotonin antagonists include 5-HT1A antagonists, 5-HT1B receptor antagonists, 5-HT1D receptor antagonists, 5-HT1F receptor antagonists, 5-HT2A receptor antagonists, 5-HT2C receptor antagonists, 5-HT4 receptor antagonists, and 5-HT7 receptor antagonists.

According to embodiments, at least one process may include a step of selecting or identifying a patient in need of treatment. According to embodiments a patient in need of treatment may be selected or identified as a patient presently diagnosed as having monoclonal gammopathy of unknown significance, premalignant bone, multiple myeloma, bone dysplasia, myeloma, amyloidosis, or myelodysplasia. According to embodiments, patients presently diagnosed may be diagnosed or identified via methods well known the skilled artisans. Such methods well known to skilled artisans may include physical examination, immunological detection methods, polymerase chain reaction (PCR)-based methods, reverse transcriptase-PCR(RT-PCR)-based methods, Southern, Northern, or Western analysis, flow cytometry, reverse phrase protein microarray (RPMA), proteomics, genomics, radiological testing processes or combinations thereof.

Embodiments relate to methods of treating monoclonal gammopathy of unknown significance (MGUS). Additional embodiments include methods of treating multiple myeloma. Still other embodiments include methods of treating myelodysplasia.

In another embodiment potential therapeutics include molecular inhibitors (e.g. Sunitinib, Dasatinib, Erlotinib), chemotherapeutics (e.g. Dexamethasone, Rapamycin, Bcl-2 inhibitor), or exogenous ligands (e.g. SCF, IGF-1 and/or cytokines (e.g. IL-6). Ideally, the potential therapeutics target a wide range of growth, prosurvival, autophagy and angiogenesis-related pathways. Exemplary candidate therapeutics include, but are not limited to, Avastin (bevacizumab), Gleevec (imatinib), Lapatinib, Iressa, Tarceva, Sutent (Sunitinib), Dasatinib (Sprycel), Nexavar (Sorafenib), Revlimid, Cucurbitacin I, A77 1726, AG 490, AG 1296, AGL 2043, Bcr-abl inhibitor, HNMPA-(AM)3, IGF-IR inhibitor, Lck inhibitor, LFM-A13, TGFβ inhibitor, CD20 antibody, Bortezomib, Carfilzomib, Chloroquine, Dasatinib, Dexamethasone, Erlotinib, Gefitinib, BCL-inhibitor, Honokiol, IGF-IR inhibitor II, Imatinib, Lapatinib, Mekl & 2 inhibitor, Melatonin, Midostaurin, Nilotinib, NVP-TKI258-CU-2, Nilotinib, Panobinostat, RAD, Rapamycin, Resveratrol, Sorafenib, Sunitinib, IL-6 ligand, IGF-1 ligand and SCF/C-kit ligand.

In another embodiment a method of treating bone disease in a subject may include administering a serotonin modulator to a subject alone or in combination with other therapies.

In another embodiment, regulating elements residing in the bone marrow may include altering the proliferation, genetic stability, survival, and/or function of cellular elements residing in bone or bone marrow.

In another embodiment, treating may include regulating bone, for example bone cells, bone marrow cells, bone stroma, gut cells, platelet cells, brain cells, or ventromedial cells.

In another embodiment, modulating of serotonin may include at least one of administering a serotonin modulator in an effective amount to modulate serotonin levels or the effects mediated by serotonin.

In another embodiment, wherein, bone breakdown inhibitors, or inhibitors that modulate osteoclast and osteoblast activity.

In another embodiment, serotonin modulator may include at least one of a tyrosine kinase inhibitor, a selective serotonin reuptake inhibitor (SSRI), a heterocyclic antidepressant, a monoamine oxidase inhibitor, an antidepressant, an anti-anxiety compound, an anti-epileptic and an antibody.

In another embodiment, treating may include treating with at least one of an agent that modulates the signaling pathways associated with the action of serotonin, an agent that is synergistic or an agent that is additive with serotonin modulation.

In embodiments cells, could be regulated by preneoplastic differentiation, neoplastic angiogenesis or stem cell function. For example bone marrow stem cell function includes differentiation of pre-osteoblasts into osteoblasts.

In another embodiment, regulating elements residing in the bone marrow may include at least one of altering the proliferation, genetic stability, survival, and/or function of cellular elements residing in bone or bone marrow.

We have developed methods of ex vivo treatment of bone marrow aspirate samples (PCT/US2009/004608). We describe methods to screen a large series of kinase and cell signaling inhibitors in fresh, living patient's myeloma cells within the tumor microenvironment within 4 hours of collection. The technology provides a method to magnetically sort, in a multiplexed high throughput manner, cellular samples with concomitant analysis of plasma cells and non-plasma cells. Employing these methods we have compiled drug inhibitory data for 35 human MM bone marrow aspirate samples using ex vivo functional screening. This information has provided insights into new therapies or combinations of therapies for treatment of MM.

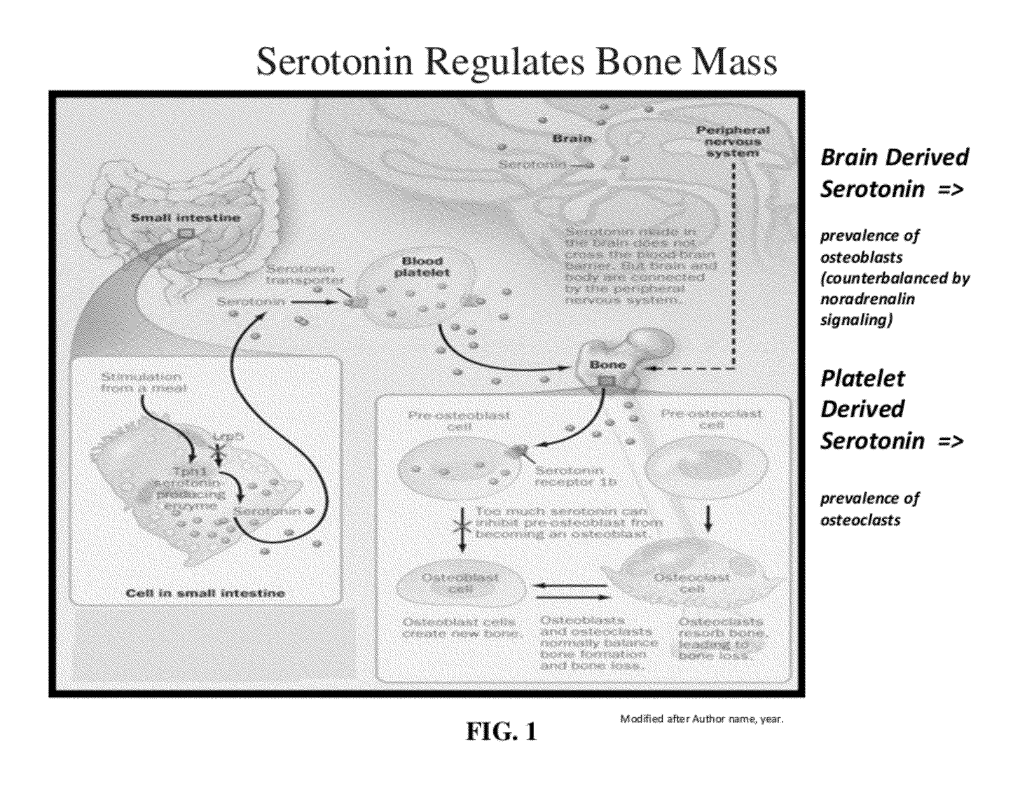

Bone marrow aspirates were treated with unique drug combinations of Chloroquine and HA14-1 (Bcl-2 inhibitor), Chloroquine and Rapamycin, or Chloroquine and tyrosine kinase inhibitors such as Sunitinib/Dasatinib/Lapatinib, etc., permitting the simultaneous evaluation of treatment effects on both myeloma (diseased) and non-diseased cells (FIG. 1). We were able to measure compensatory up-regulation of cell signaling pathways by reverse phase protein microarray as a prognostic indicator of drug resistance. In addition this method allowed the differential effect of treatment on the CD138+ and non-CD138+ cell populations to be quantitated. This method may be used for determining toxicity on normal cells in individual patients for therapeutic decisions.

We propose a means of treating any stage of multiple myeloma (where the myeloma cells are growth inhibited, suppressed, or killed, to a greater extent compared to the non-myeloma cells) with the combination of an autophagy inhibitor with a non-chemotherapeutic agent (such as a tyrosine kinase inhibitor, small molecule inhibitor or a therapeutic antibody with examples listed in Table 1 below). We also propose the combination of an autophagy inhibitor with non-chemotherapeutic agents for the treatment of patients with myeloma pre-cursors diseases, Monoclonal Gammopathy, Multiple Gammopathy of Unknown origin Syndrome (MGUS), amyloidosis, plasmacytoma, or any other plasma cell related disease.

This work can quantitatively measure the phosphorylation, cleavage or total forms of kinases, phosphatases and other cell signaling proteins in bone marrow aspirate and bone marrow core samples for treatment regimen stratification. Specific inhibitors, such as gefitinib, erlotininb, and surafinib, or combinations of inhibitors with steroids (dexamethasone) and/or autophagy inhibitors can be tested ex vivo using a patient's bone marrow aspirate to predict which patient will respond to a particular therapy or combination. The multiplexed nature of the reverse phase protein microarray technology permits quantitative measurement of multiple cell signaling proteins. This work can be used to generate a functional multiple myeloma or leukemia classifier based on drug target activation and test the hypothesis that cell signaling activation portraits can predict a priori which targeted therapies will best cause cell death.

This work can provide simultaneous assessments of treatment effects on diseased and non-diseased cell populations. For example, Non-plasma cells and plasma (myeloma) cells can be concomitantly studied for therapeutic efficacy for an individual patient. Analysis of both diseased and non-diseased cell populations, under the same conditions, with the same treatments, can be used to predict potential toxicity as well as efficacy.

While not intending to be bound to any particular mechanism, Applicants propose a mechanism in FIG. 1. The biologic mechanisms involved in the pathogenesis of multiple myeloma (MM)-induced osteolytic bone disease are less well understood. Physiological interactions between the serotoninergic and skeletal systems are implicated by clinical observations [1]. The RPMA used in this invention has revealed a new role for serotonin signaling in myeloma/MGUS osteolytic bone disease.

We propose a means of treating or preventing brittle bone disease or osteolytic bone disease which comprises a serotonin modulator alone, or in combination with, an autophagy inhibitor and/or a non-chemotherapeutic agent (examples listed in Table 1 below).

The monoamine serotonin [5-hydroxytryptamine (5-HT)] has previously been investigated as a neurotransmitter, synthesized by a two-step pathway in which tryptophan hydroxylase is the rate-limiting enzyme. Circulating 5-HT is principally stored in platelet-dense granules. Aggregated immunoglobulins derived from all the IgG subclasses, isolated from healthy controls or myeloma patients, induce platelet granules release in the absence of antigen or particulate matter, in a dose dependent manner [2].

The brainstem-derived serotonin (BDS) positively regulates bone mass following binding to 5-HT2C receptors on ventromedial hypothalamic neurons. This is opposed by platelet-derived serotonin (PDS) which induces bone lysis and osteoclast activation.

Immunoglobulins have been shown to induce platelet release a) when participating in immune reactions as antigen-antibody complexes or b) by nonimmune mechanisms such as coating of glass or polymethylmethacrylate beads.

MM patients with evidence of osteolytic lesions exhibited an increase in the concentration of serum tryptophan and serotonin [3], while that of tyrosine, dopamine, and noradrenaline was decreased [3].

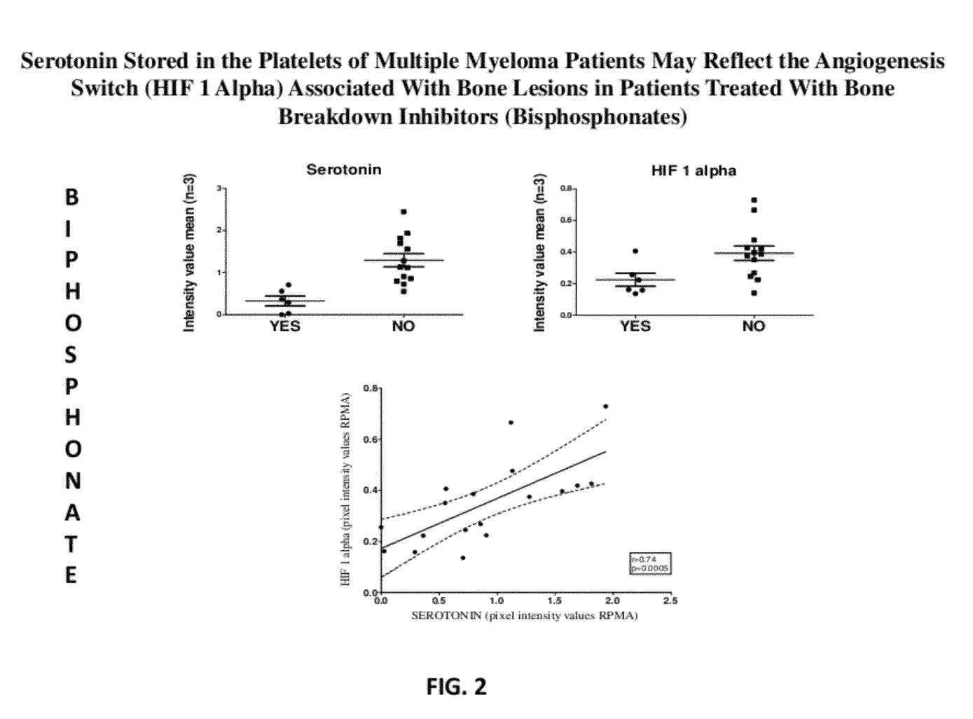

We found that bone marrow cells from patients with osteolytic multiple myeloma has higher levels of serotonin, RANK, Beta Arrestin and DEPTRO compared to non-osteolytic myeloma patients (FIG. 2). Increased circulating-serotonin levels released from platelets by immunoglobulin complexes may alter the RANK/RANKL ratio in the BM environment and promote MM osteolytic lesion.

These data indicate that the 5-HT system plays an important role in bone homeostasis through effects on osteoclast function and that the serotonin system is involved in the pathogenesis of MM-induced bone disease. Therefore serotonin regulation is a new therapeutic target for preventing or treating osteolytic bone disease associated with multiple myeloma or other conditions.

The following references are included provide background information as an aid to explain the present embodiments:

In this specification, “a” and “an” and similar phrases are to be interpreted as “at least one” and “one or more.”

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described exemplary embodiments. In particular, it should be noted that, for example purposes, the above explanation has focused on the example(s) serotonin modulators. However, one skilled in the art will recognize that embodiments of the invention could be serotonin agonists, serotonin antagonists, or both.

In addition, it should be understood that any figures which highlight the functionality and advantages, are presented for example purposes only. The disclosed architecture is sufficiently flexible and configurable, such that it may be utilized in ways other than that shown. For example, the steps listed in any flowchart may be re-ordered or only optionally used in some embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112, paragraph 6. Claims that do not expressly include the phrase “means for” or “step for” are not to be interpreted under 35 U.S.C. 112, paragraph 6.

### Example 1

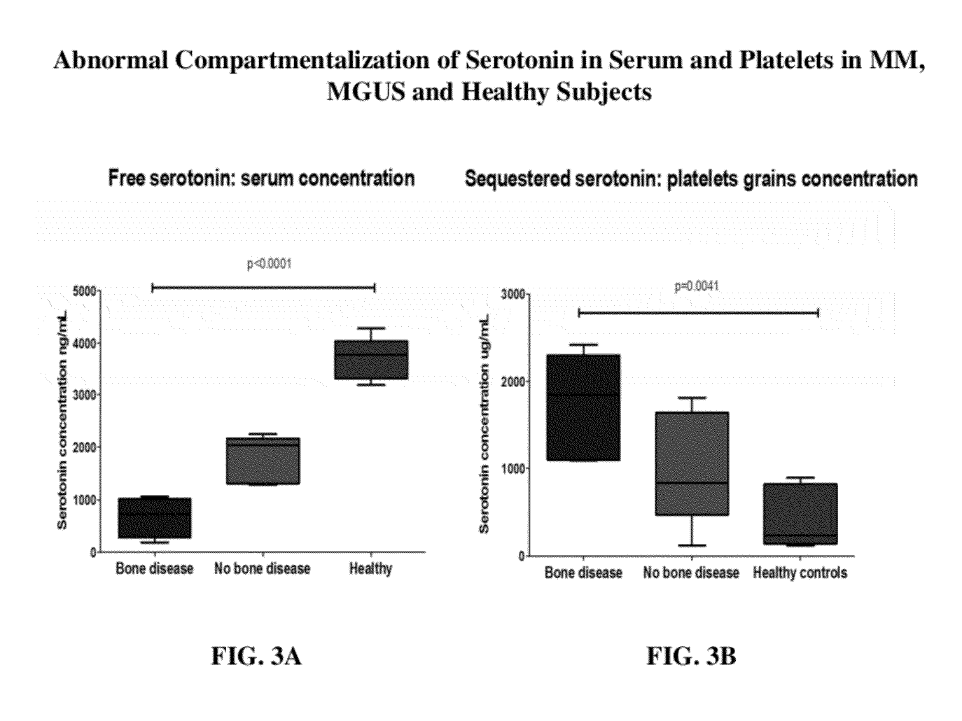

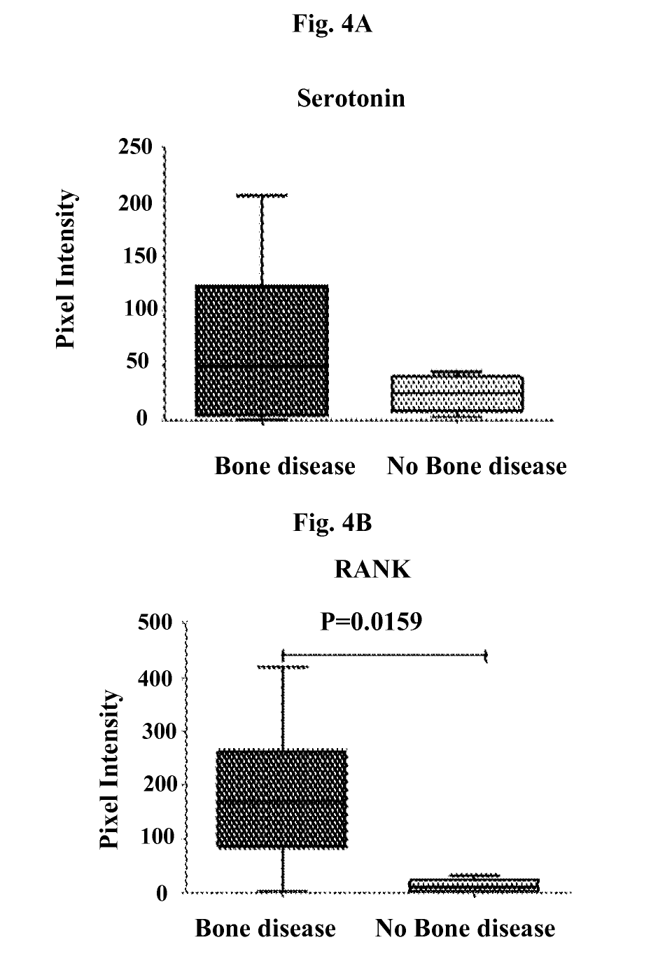

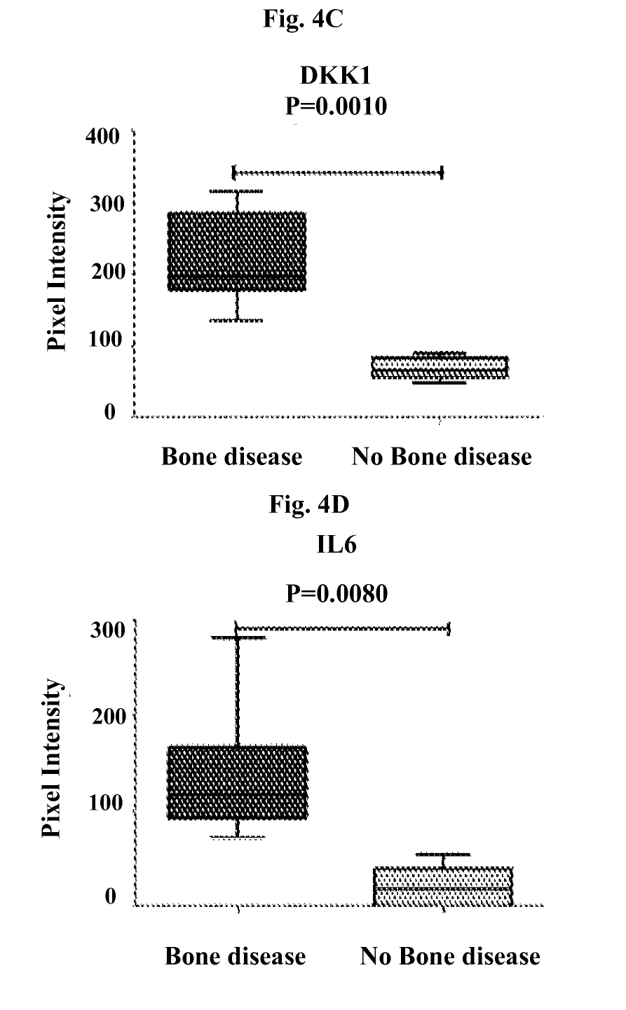

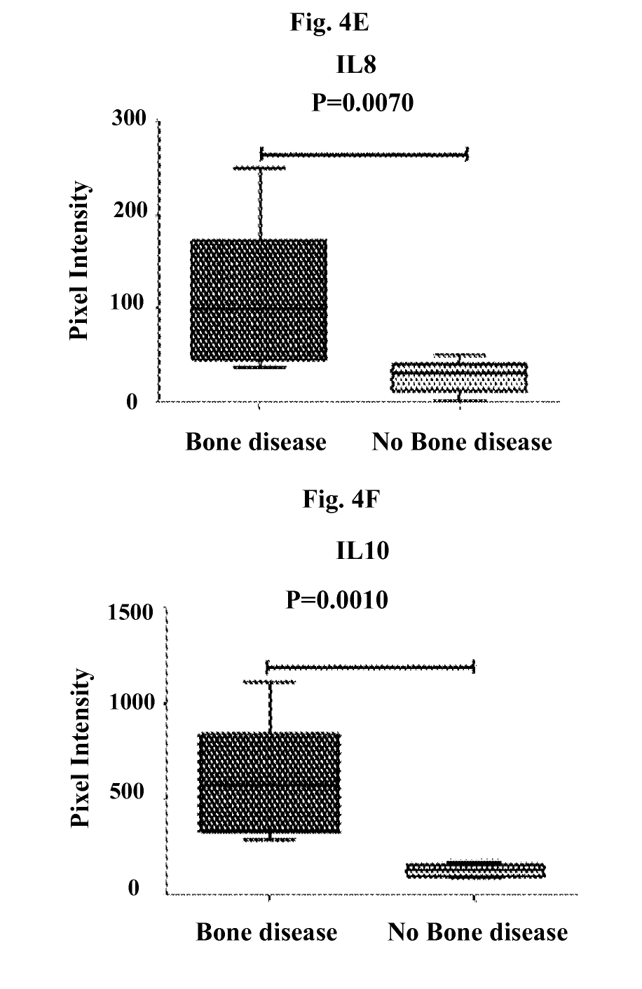

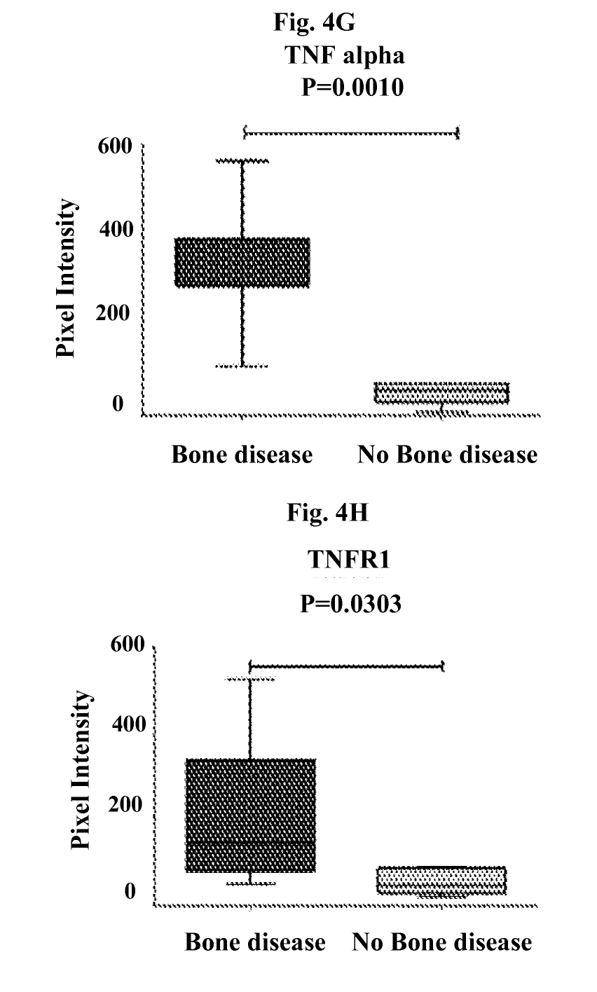

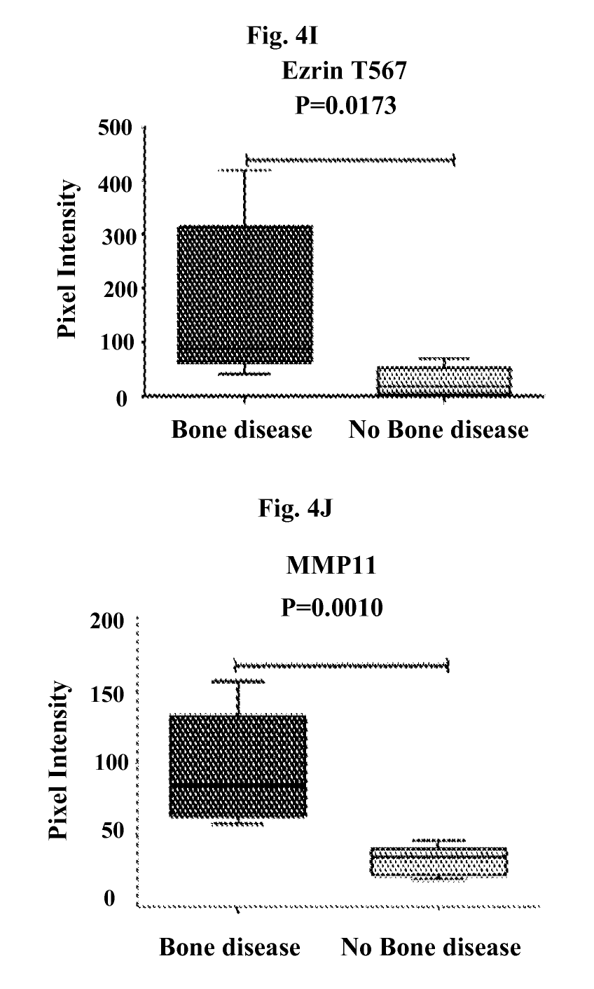

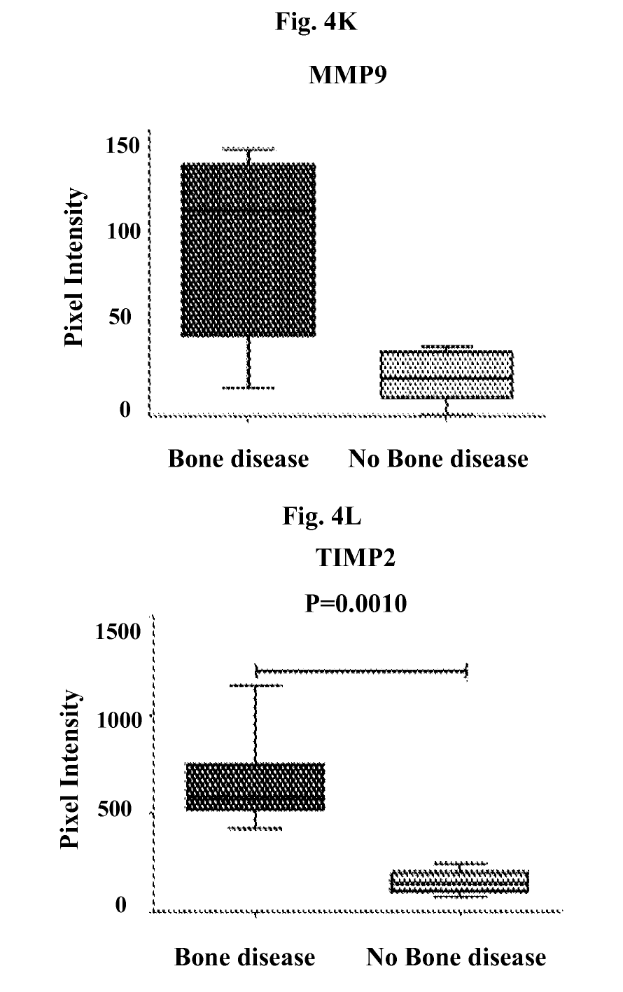

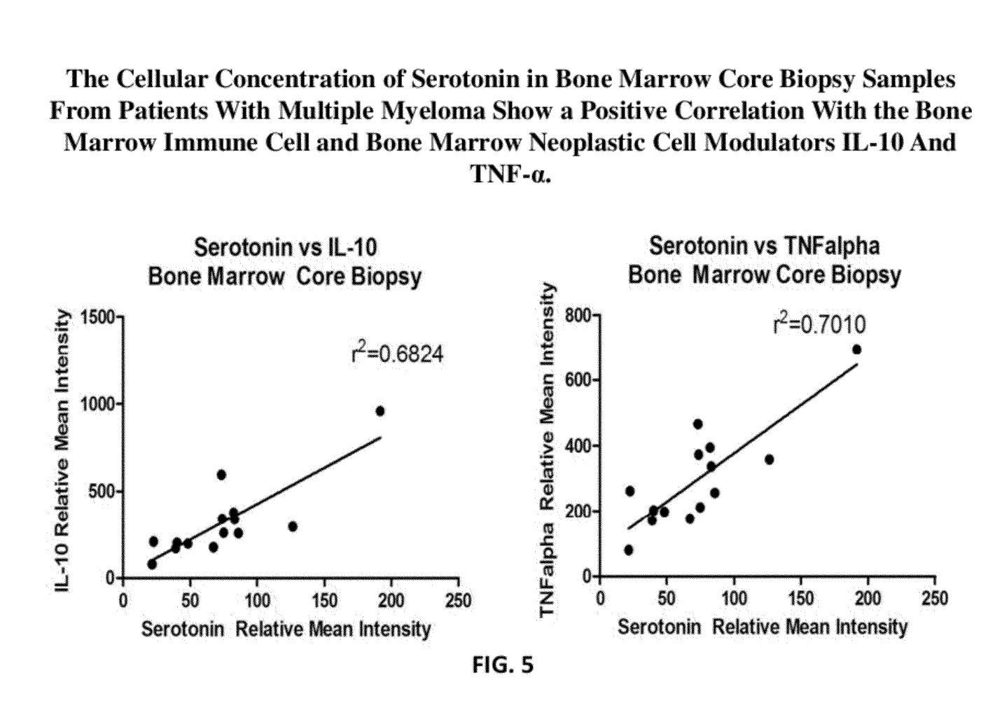

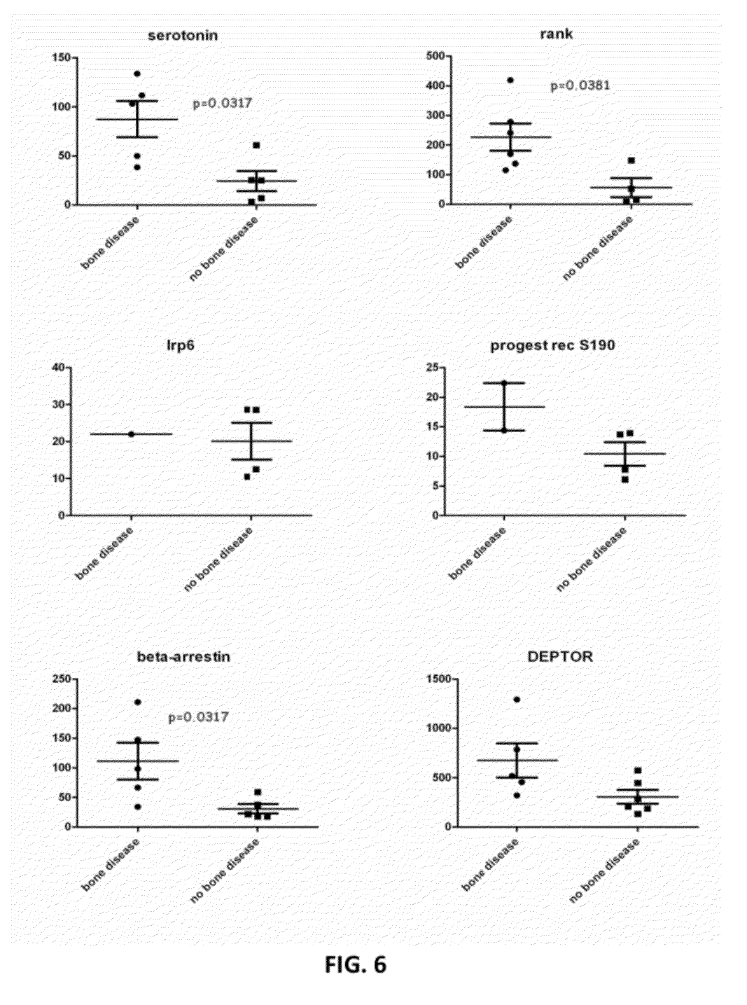

Bone marrow aspirates were treated with unique drug combinations of Chloroquine and HA14-1 (Bcl-2 inhibitor), Chloroquine and Rapamycin, or Chloroquine and tyrosine kinase inhibitors such as Sunitinib/Dasatinib/Lapatinib, etc., permitting the simultaneous evaluation of treatment effects on both myeloma (diseased) and non-diseased cells (FIG. 1). Compensatory up-regulation of cell signaling pathways was measured by reverse phase protein microarray as a prognostic indicator of drug resistance. In addition this method allowed the differential effect of treatment on the CD138+ and non-CD138+ cell populations to be quantitated. This method may be used for determining toxicity on normal cells in individual patients for therapeutic decisions. The results are shown in FIGS. 2-6.

TABLE 1
Autophagy combination therapy example agents.

Inhibitors
17-DMAG
8-hydroxy Guanosine
AKT Inhibitor IV
AKT inhibitor X
AKT inhibitor XI
AMPK Inhibitor, Compound C
BAY 11-7082
Bcr-abl Inhibitor
Bortezomib
Carfilzomib
Caspase-3 Inhibitor VII
Caspase-8 inhibitor I
Caspase-9 inhibitor II
CGP041251 (Midostaurin)
Chloroquine
Cox II Inhibitor
Dasatinib
Dexamethasone
EGFR inhibitor II, BIBX1382
EGFR/Erb-2/Erb-4 Inhibitor
ERK inhibitor II, Negative control
ERK inhibitor III
erlotinib
FGF/VEGF Receptor Tyrosine Kinase Inhibitor, PD173074
Gefitinib
Glycogen Phosphorylase Inhibitor
Granzyme B inhibitor I
HA14-1
HNMPA-(AM)₃ (Insulin Receptor TKI inhibitor)
Honokoil
HSP90 Inhibitor
IGF-1R Inhibitor II
IGF-1R PPP
Imatinib
Imatinib
Jak2 Inhibitor II
Jak3 Inhibitor I
JNK Inhibitor I, (L)-Form
K2529
Lapatinib
LY294002
MAPK Inhibitor PD169316
Mek 1 & 2 inhibitor SL327
Melatonin
Melphalan
NVP-BEZ235
NVP-Raf-265
NVP-LBH589
NVP-AMN107 (Nilotinib)
NVP-TKI258-CU-2
PARP Inhibitor XI, DR2313
PD153035 (EGFR Inhibitor)
PD98059 (MEK inhibitor)
PDGF Receptor Tyrosine Kinase Inhibitor I
PI 3-Kα Inhibitor IV
PI 3-Kγ Inhibitor II
Proteasome Inhibitor IX, AM114
RAD001
Rapamycin
Resveratrol
Sorafinib
Src Kinase Inhibitor II
Sunitinib
Terphenyl (FWF416)
VEGF Receptor Tyrosine Kinase Inhibitor III, KRN633
Wortmannin
ZM 336372 (c-Raf inhibitor)
hydroxychloroquine
3-methyladenie
clomipramine
ethyl pyruvate
glycyrrhizin
Asparagine (Asn)
Leupeptin
Serotonin or serotonin related inhibitor
Serotonin modulator agents such as serotonin reuptake
inhibitors, or serotonin receptor antagonists
Bisphosphonates and other nitrogenous or non-nitrogenous
inhibitors such as Clodronate or Zoledronic Acid
Collagenase inhibitors such as Matrix Metalloproteinase
Inhibitors

### REFERENCES

1. Rosen, Nature Medicine (2009), 15:2, 145-6
2. Zimmermann, The Journal of Clinical Investigation (1975), 56, 828-834
3. Kurup, International Journal of Neuroscience, (2003), 113:9, 1221-1240.

What is claimed is:

1. A method of treating bone disease comprising administering a serotonin modulator to a subject.

2. The method of claim 1, wherein said treating comprises at least one of the following:
a. increasing bone density;
b. decreasing bone density;
c. maintaining bone density; and
d. regulating elements associated with bone marrow.

3. A method of claim 2 where said regulating comprises altering at least one of the following: preneoplastic differentiation of bone marrow cells, neoplastic angiogenesis of bone marrow cells, or bone marrow stem cell function.

4. The method of claim 1, wherein said serotonin modulator alters at least one of the following:
a. ratio of serotonin in platelets to plasma;
b. concentration of serotonin in the bone marrow;
c. serotonin receptor activity of cells within the bone marrow;
d. production of serotonin by cells associated with the bone;
e. intracellular signaling pathways associated with serotonin;

5. The method of claim 4 wherein said serotonin modulator comprises at least one of the following:
a. a tyrosine kinase inhibitor;
b. a selective serotonin reuptake inhibitor (SSRI);
c. a heterocyclic antidepressant;
d. a monoamine oxidase inhibitor;
e. an antidepressant;
f. an anti-anxiety compound;
g. an anti-epileptic; and
h. an antibody.

6. The method of claim 5 wherein the monoamine oxidase inhibitor is a selective monoamine oxidase inhibitor, a monoamine oxidase A inhibitor, a monoamine oxidase B inhibitor or a nonselective monoamine oxidase inhibitor.

7. The method of claim 1, further comprising administering at least one of:
a. an autophagy inhibitor;
b. a non-chemotherapeutic agent;
c. an angiogenesis inhibitor;
d. a bone breakdown inhibitor;
e. a osteoclast or osteoblast activity inhibitor; and
f. an immune signal modulator.

8. The method of claim 1, further comprising reducing serotonin from platelet cells, gastrointestinal cells, neural cells, immune cells, bone marrow microenvironment cells or cancer cells.

9. The method of claim 7, wherein said “bone breakdown inhibitor” is administered in an effective amount to modulate at least one of bone cell activity, stem cell activity, gastrointestinal cell activity, cancer cell activity, platelet cell activity, and neural cell activity.

10. The method of claim Error! Reference source not found., wherein said bone disease comprises at least one of:
a. brittle bone disease;
b. multiple myeloma;
c. osteogenesis imperfecta (OI)
d. osteolytic bone disease;
e. amyloidosis;
f. monoclonal gammopathy;
g. alterations in bone marrow hematopoetic precursor cells; and
h. myelodysplasia.

11. The method of claim 10 wherein said subject may include at least one of a chordate, mammal, primate, and human.

12. The method of claim 10 wherein myeloma cells are inhibited, suppressed, or killed to a greater extent as compared to the non-myeloma cells.

13. The method of claim 1, further comprising selecting a subject in need of treatment.

14. A method of diagnosing a subject for a bone disease comprising:
a. assaying a biological sample of the subject;
b. determining the amount of serotonin in said biological sample; and
c. determining a disease state based on said amount of serotonin.

15. The method of claim 14, wherein said biological sample comprises at least one of the following:
a. bone marrow aspirate;
b. tissue;
c. blood serum;
d. whole blood;
e. cells; and
f. blood.

16. The method of claim 14, wherein said determining comprises:
a. determining the amount of serotonin in a known normal sample;
b. determining the amount of serotonin in said biological sample;
c. comparing said amount of serotonin in said known normal sample to the amount of serotonin in said biological sample.

17. The method of claim 14, further comprising treating said subject based on said determination.

18. The method of claim 14, further comprising regulating bone remodeling in the subject including modulating serotonin in the subject based on said diagnosing.

19. The method of claim 14, wherein said assaying step comprises assays evaluating post translational modification of signaling proteins, caspase cleavage, poly(ADP-ribose) polymerase (PARP) cleavage or dye exclusion/uptake.

20. The method of claim 19, wherein said evaluating is selected from the group consisting of reverse phase microarray (RPMA), ELISA, flow cytometry, Immunohistochemistry, Immunoassay, high resolution mass spectroscopy, and suspension bead array.

21. A method of treating monoclonal gammopathy of unknown significance (MGUS), premalignant bone marrow cells or multiple myeloma in a subject, comprising administering:
a. an autophagy pathway inhibitor; and
b. at least one of a tyrosine kinase inhibitor, a serotonin modulator, an antidepressant, an anti-anxiety compound, an antiepileptic, a monoamine oxidase inhibitor, an antibody, a non-chemotherapeutic agent and a bisphosphonate.

22. The method of claim 21, wherein said autophagy pathway inhibitor is a 4-amino quinoline.

23. The method of claim 21 wherein said bisphosphonate may include at least one of alendronate, pamidronate or zoledronic acid.

24. The method of claim 21, wherein said treatment retards the progression from a pre-disease state to multiple myeloma.
